(19) United States
(12) Patent Application Publication
(10) Pub. No.: US 2010/0137681 A1
(43) Pub. Date: Jun. 3, 2010

(54) ENDOSCOPIC INSTRUMENT MANAGEMENT SYSTEM

(75) Inventors: Richard C. Ewers, Fullerton, CA (US); Christopher James Earley, San Clemente, CA (US); Barton P. Bandy, Orange, CA (US); Haio Fauser, Encinitas, CA (US)

Correspondence Address: LEVINE BAGADE HAN LLP, 2400 GENG ROAD, SUITE 120, PALO ALTO, CA, 94303, US

(73) Assignee: USGI Medical, Inc., San Clemente, CA (US)

(21) Appl. No.: 12/623,224

(22) Filed: Nov. 20, 2009

Related U.S. Application Data
(60) Provisional application No. 61/116,955, filed on Nov. 21, 2008.

(51) Int. Cl. A61B 1/01; A61B 1/018

(52) U.S. Cl. 600/102; 600/106

(57) ABSTRACT

Endoscopic instrument management systems are described herein which allow one or more operators to manage multiple different instruments utilized in endoscopic procedures. In one aspect, responsibility for instrumentation management between one or more operators may be configured such that a first set of instruments is controlled by a primary operator and a second set of instruments is controlled by a secondary operator. The division of instrumentation may be facilitated by the use of separated instrumentation platforms or a single platform which separates each instrument for use by the primary operator. Such platforms may be configured as trays, instrument support arms, multi-instrument channels, as well as rigidized portions of instruments to facilitate its handling, among others. In another aspect, one or more plastically deformable instrument manifolds are provided to guide flexible endoscopic instruments into and through an endoscopic access device.

### CROSS-REFERENCE TO RELATED APPLICATION

This application claims benefit of priority to U.S. Provisional Patent Application No. 61/116,955, filed on Nov. 21, 2008, the content of which is incorporated herein by reference in its entirety. This application also relates to U.S. patent application Ser. No. 12/138,348 (Attorney Docket No. USGINZ05600), filed Jun. 12, 2008, the content of which is also incorporated herein by reference in its entirety.

### FIELD OF THE INVENTION

The present invention relates to methods and apparatus for managing one or more instruments and/or tools used during endoscopic diagnostic and therapeutic procedures. More particularly, the present invention relates to methods and devices used to facilitate instrument management and use during procedures where flexible endoscopic instruments are advanced into a patient body via one or more natural orifices or other access ports.

### BACKGROUND OF THE INVENTION

Endoscopic procedures and surgery typically entail the advancement and use of one or more instruments through the natural orifices and/or other access ports of a patient body and through the tortuous endoscopic pathways to reach the tissue regions of interest. Even procedures performed in body spaces within the patient may entail entry and advancement through one or more openings created in the patient body to gain entry into the desired body space, e.g., entry through a percutaneous opening or a gastrotomy to gain entry into the peritoneal space of the patient.

Because endoscopic surgery may involve the use of multiple instruments through a single conduit into the patient body, efficient management and use of these instruments may be difficult in part not only because of the number of instruments utilized, but also because these multiple instruments typically converge from a single conduit, which may be limited by the cross-sectional profile of the body lumen, organ, orifice, passageway, etc., in which the conduit is disposed. At the same time, advances in therapeutic endoscopy have led to an increase in the complexity of endoscopic operations attempted, as well as the complexity of tools advanced through the working lumens of these conduits.

Because of the number of instruments which converge typically from a single conduit, difficulties may arise in effectively handling and managing the placement, positioning, and use of these multiple instruments in an effective and safe manner.

For example, flexible endoscopes and flexible endoscopic instruments provide the ability for an operator to intubate the patient and to provide therapy to the internal anatomy by way of non-straight access pathways. Typical endoscopes have the ability to steer at the tip and provide light and visualization, gas insufflation, and lens rinsing. Such endoscopes will typically include one or two instrument channels. These instrument channels include an angled interface on the handle of the endoscope having a bend of about 45 degrees on a relatively short section of the handle. One result of this configuration is that any instrument that is to be inserted into the endoscope instrument channel must include a shaft that is flexible over its entire length.

Accordingly, there is a need for methods and devices for facilitating the introduction and management of all the instruments advanced through the relatively small conduits for performing endoscopic procedures.

### SUMMARY OF THE INVENTION

An endoscopic tissue manipulation assembly may comprise, at least in part, a distal end effector assembly disposed or positionable at a distal end of a flexible and elongate body. Examples are described in further detail in U.S. Pat. Pub. No. 2005/0272977 A1, which is incorporated herein by reference in its entirety. A handle assembly may be connected to a proximal end of the elongate body and include a number of features or controls for articulating and/or manipulating both the elongate body and/or the distal end effector assembly. The elongate body may optionally utilize a plurality of locking or lockable links nested in series along the length of the elongate body which enable the elongate body to transition between a flexible state and a rigidized or shape-locked configuration. Details of such a shape-lockable body may be seen in further detail in U.S. Pat. Nos. 6,783,491; 6,790,173; and 6,837,847, each of which is incorporated herein by reference in its entirety.

One or more various instruments may be passed through the elongate body for deployment through its distal end by introducing the instruments through one or more corresponding tool ports located in the handle assembly. One instrument in particular which may be used to endoscopically visualize procedures and tissue regions of interest may include an endoscope or imaging system having a flexible shaft which may be introduced into the elongate body via a side port, e.g., Y-Port, located along the elongate body and distal to the handle assembly.

Because of the number of different instruments and the different types of tools which may be utilized in the endoscopic tissue manipulation assembly, tool or instrumentation management is one consideration for the practitioner or practitioners to facilitate efficient surgical and/or endoscopic procedures when performed upon a patient. Additionally, the division of responsibility for instrumentation management between one or more practitioners is highly desirable to ensure patient safety and procedure facilitation. Table-mounted or stand-alone instrument support members, such as instrument clamps, stands, or other devices may be used to assist with management of endoscopic access devices, tools, and/or instruments.

Aside from table-mounted or stand-alone instrument supporting members, additional instrument management systems may be employed which a single operator or user may utilize. In a first aspect, a multi-instrument support arm extending proximally from the handle assembly generally comprises a stiffened multi-lumen channel having a straight support channel extending proximally and one or more angled or curved support channels projecting at an angle therefrom support arm. Because the multi-instrument support arm is relatively stiff, it may be engaged to the handle assembly and used to support and separate its respective instruments leaving the operator to hold a single handle during a procedure. Other variations include a pivoting multi-instrument support having one or more individual instrument ports pivotably positioned within an open channel. Still other variations include a manifold that is attachable to the handle assembly and that supports one or more elongated straight docking sections each defining a substantially straight lumen for receiving an instrument shaft in a slidable docking configuration.

Another method for facilitating instrument management utilizes forming rigid portions of the instrument shafts. The elongate shaft is generally configured as a flexible length so as to traverse through the elongate body and within the patient body via endoluminal pathways. In another aspect, a portion of the elongate shaft extending between the handle and flexible length is configured as a rigid section, and may include a rigid sleeve made, e.g., from stainless steel or some other rigid metal or polymer, which is formed over the portion of the shaft extending from the handle. Alternatively, the rigid portion is formed integrally with the elongate shaft, e.g., as a section reinforced by woven metallic braids or inserts. In use, the flexible length of the elongate shaft is advanced through a tool port and through the handle assembly. The rigid section extending from the handle is advanced at least partially into the tool port such that the handle is supported or held in a linear configuration relative to the tool port and handle assembly by the rigid section.

The interface between the rigid portion(s) of the instrument shaft(s) and the straight sections of the tool port(s) provided in the handle assembly provides the operator with the ability to slidably dock the instruments within the endoscopic access device. The slidable docking interface provides several benefits. For example, the operator is able to release the instrument to use his hand for other purposes without having the instrument drop or flop downward, as would be the case with a flexible shafted instrument. In addition, the slidable docking interface facilitates instrument management using only a single support arm for the endoscopic access device, rather than requiring separate support for each instrument inserted into the device. Further, rigid shafted instruments provide improved force transmission and the slidable docking interface reduces or eliminates the possibility that an exposed shaft will bend or buckle. Still further, having a substantially straight tool port lumen in the handle assembly retains the ability to use flexible shafted instruments, if desired. Finally, having a substantially straight tool port lumen in the handle assembly facilitates insertion of instruments having longer rigid working lengths and/or larger shaft diameters. For example, a typical endoscope has an instrument channel with an inlet having a 45 degree bend. All tools used in the channel must be sufficiently flexible to pass the 45 degree bend. Having a substantially straight lumen provides the ability to use many instruments that could not be used through the instrument channel of a conventional endoscope.

Another variation of the instrument management system includes the provision of a flexible joint or flexible section of the instrument shaft between the handle and a rigid proximal section of the shaft. The flexible joint/section allows the handle to be flexed away from other instruments but retain sufficient rigidity that the handle does not droop. In this manner, the instrument handles are able to be flexed apart to prevent or reduce clashing.

In still another aspect, an endoscopic instrument management manifold is attachable to the handle assembly and provides one or more elongated pathways for passage of a flexible instrument shaft. In several embodiments, the one or more elongated pathways are defined by one or more extension tubes that extend from the proximal end of the handle assembly. In several other embodiments, the elongated pathways are defined by one or more extension tubes that are capable of being manipulated to assume a desired shape or orientation.

### BRIEF DESCRIPTION OF THE DRAWINGS

FIGS. 1A and 1B shows assembly and end views, respectively, of an endoscopic tissue manipulation system and examples of the various endoscopic instruments which may be advanced therethrough.

FIG. 2 shows the endoscopic manipulation system of FIG. 1A disassembled into its separate instrument components for illustrative purposes.

FIGS. 3 to 5 illustrate side views of a tissue manipulation assembly operable via a launch tube member which may be advanced through the endoscopic system.

FIGS. 6 and 7 illustrate perspective and top views, respectively, of a stiffened multi-instrument support arm having one or more angled or curved support channels projecting therefrom.

FIGS. 8 and 9 illustrate perspective and top views, respectively, of another stiffened multi-instrument support arm having a straight tubular member and one or more angled or curved support channels.

FIGS. 10A to 10C illustrate perspective, top, and end views, respectively, of another variation for a pivoting multi-instrument support having a fanned or angled lumen enclosure.

FIGS. 11 and 12 show top views illustrating examples for altering the entry lumen angle of the individual instrument ports.

FIGS. 13 and 14 show perspective views of a manifold supporting a pair of elongated docking sections that is attachable to the proximal end of an endoscopic access device.

FIGS. 15A to 15C illustrate side views of an instrument management system utilizing rigid portions of an instrument shaft for providing support to the instrument projecting from a handle assembly.

FIGS. 16 and 17 show end and top views, respectively, of tool ports having tapered entries for facilitating the insertion of instruments therethrough.

FIGS. 18A and 18B show exploded and perspective views of a rotating clamp adapted to be attached to an endoscopic access device.

FIGS. 19A and 19B show an endoscopic device having a straight, elongated docking lumen formed in the handle, and an instrument having a rigid shaft section near its proximal end.

FIGS. 20A and 20B show top views of an endoscopic device handle assembly having a plurality of instruments extending from its proximal end.

FIGS. 21A and 21B show top views of a physician using an endoscopic device during a procedure being performed on a patient.

FIGS. 22A and 22B are side views of an endoscopic device having a pair of endoscopic instrument management manifolds.

FIGS. 23A and 23B are a side view and an endoscopic view, respectively, of an endoscopic access device having instruments extending therethrough.

FIGS. 23C, 23D, and 23E are a side view and two endoscopic views, respectively, of the device of FIGS. 23A and 23B in a retroflexed position.

FIGS. 24A and 24B are a side view and an endoscopic view, respectively, of an endoscopic access device in a retroflexed orientation and having instruments extending through endoscopic instrument management manifolds that are also in a retroflexed orientation.

FIGS. 25A and 25B are a side view and an endoscopic view, respectively, of an endoscopic access device in a retroflexed orientation and having instruments extending through endoscopic instrument management manifolds that are in a crossed-over orientation.

FIG. 26 is a cross-sectional view of a telescoping endoscopic instrument management manifold.

FIGS. 27A to 27C are side views of an instrument, a manifold, and an instrument inserted into a manifold, respectively, illustrating an interlocking feature.

### DETAILED DESCRIPTION OF THE INVENTION

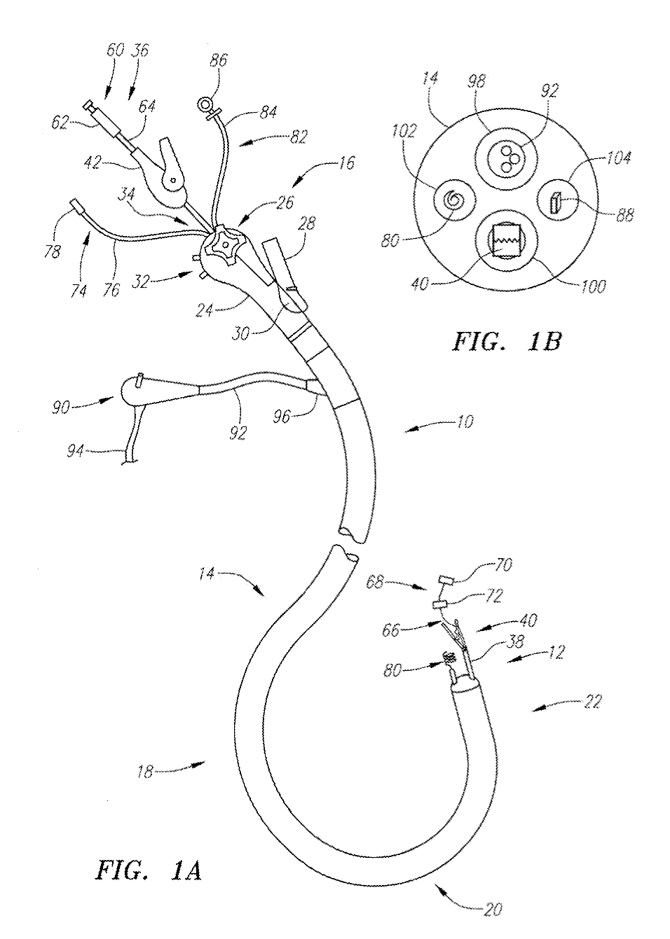

With reference to FIG. 1A, the endoscopic tissue manipulation system 10 as described herein may comprise, at least in part, a distal end effector assembly 12 disposed or positionable at a distal end of a flexible and elongate body 14. Examples of the tissue manipulation system 10 are described in further detail in U.S. Pat. Pub. No. 2005/0272977 A1, which is incorporated herein by reference in its entirety. Additional examples of endoscopic access devices and systems incorporating such devices are described in further detail in U.S. patent application Ser. No. 12/061,951, filed Apr. 2, 2008, which is also incorporated herein by reference in its entirety. A handle assembly 16 may be connected to a proximal end of the elongate body 14 and include a number of features or controls 26 for articulating and/or manipulating both the elongate body 14 and/or the distal end effector assembly 12.

As shown, the system 10 may comprise a number of various instruments and devices utilized in various combinations with one another to effect any number of different procedures. Accordingly, each of the instruments and devices may require manipulation or some degree of handling by the practitioner.

The elongate body 14 may optionally utilize a plurality of locking or lockable links nested in series along the length of the elongate body 14 which enable the elongate body 14 to transition between a flexible state and a rigidized or shape-locked configuration. Details of such a shape-lockable body may be seen in further detail in U.S. Pat. Nos. 6,783,491; 6,790,173; and 6,837,847, each of which is incorporated herein by reference in its entirety. Alternatively, elongate body 14 may comprise a flexible body which is not rigidizable or shape-lockable but is flexible in the same manner as a conventional endoscopic body, if so desired. Additionally, elongate body 14 may also incorporate additional features that enable any number of therapeutic procedures to be performed endoscopically. Elongate body 14 may be accordingly sized to be introduced per-orally. However, elongate body 14 may also be configured in any number of sizes, for instance, for advancement within and for procedures in the lower gastrointestinal tract, such as the colon.

Elongate body 14, in one variation, may comprise several controllable bending sections along its length to enable any number of configurations for the elongate body 14. Each of these bending sections may be configured to be controllable separately by a user or they may all be configured to be controlled simultaneously via a single controller. Moreover, each of the control sections may be disposed along the length of elongate body 14 in series or they may optionally be separated by non-controllable sections. Moreover, one, several, or all the controllable sections (optionally including the remainder of elongate body 14) may be rigidizable or shape-lockable by the user.

In the example of endoscopic tissue manipulation system 10, elongate body may include a first articulatable section 18 located along elongate body 14. This first section 18 may be configured via handle assembly 16 to bend in a controlled manner within a first and/or second plane relative to elongate body 14. In yet another variation, elongate body 14 may further comprise a second articulatable section 20 located distal of first section 18. Second section 20 may be configured to bend or articulate in multiple planes relative to elongate body 14 and first section 18. In yet another variation, elongate body 14 may further comprise a third articulatable section 22 located distal of second section 20 and third section 22 may be configured to articulate in multiple planes as well, e.g., 4-way articulation, relative to first and second sections 18, 20.

As mentioned above, one or each of the articulatable sections 18, 20, 22 and the rest of elongate body 14 may be configured to lock or shape-lock its configuration into a rigid set shape once the articulation has been desirably configured. Detailed examples of such an apparatus having one or multiple articulatable bending sections which may be selectively rigidized between a flexible configuration and a shape-locked configuration may be seen, e.g., in U.S. Pat. Pub. Nos. 2004/0138525 A1, 2004/0138529 A1, 2004/0249367 A1, and 2005/0065397 A1, each of which is incorporated herein by reference in its entirety. Although three articulatable sections are shown and described, this is not intended to be limiting as any number of articulatable sections may be incorporated into elongate body 14 as practicable and as desired. Moreover, one or multiple sections may be comprised of a series of nested-links which allow the one or more sections 18, 20, 22 to be articulated or deflected relative to one another along their lengths and optionally rigidized to conform and hold any particular shape.

Handle assembly 16 may be attached to the proximal end of elongate body 14 via a permanent or releasable connection. Handle assembly 16 may generally include a handle grip 24 configured to be grasped comfortably by the user and an optional rigidizing control 28 if the elongate body 14 and if one or more of the articulatable sections are to be rigidizable or shape-lockable. Rigidizing control 28 in this variation is shown as a levered mechanism rotatable about a pivot 30. Depressing control 28 relative to handle 24 may compress the internal links within elongate body 14 to thus rigidize or shape-lock a configuration of the body while releasing control 28 relative to handle 24 may in turn release the internal links to allow the elongate body 14 to be in a flexible state. Further examples of rigidizing the elongate body 14 and/or articulatable sections may again be seen in further detail in U.S. Pat. Pub. Nos. 2004/0138525 A1, 2004/0138529 A1, 2004/0249367 A1, and 2005/0065397 A1, incorporated above by reference. Although the rigidizing control 28 is shown as a lever mechanism, this is merely illustrative and is not intended to be limiting as other mechanisms for rigidizing an elongate body, as generally known, may also be utilized and are intended to be within the scope of this disclosure.

Handle assembly 16 may further include a number of articulation controls 26, as described in further detail below, to control the articulation of one or more articulatable sections 18, 20, 22. Handle 16 may also include one or more ports 32 for use as insufflation and/or irrigation ports, as so desired.

Furthermore, one or more various instruments may be passed through elongate body 14 for deployment through distal end 12 by introducing the instruments through one or more corresponding tool ports 34 located in handle assembly 16. As mentioned above, a number of different endoscopic and/or endoluminal instruments having a flexible body may be delivered through system 10 to effect any number of endoscopic procedures.

One example of such an instrument may include an endoscopic tissue manipulation and securement assembly 36, as described in further detail below, which may be introduced into system 10 via instrument lumen 100, as shown in the end view of distal end 12 in FIG. 1B. Any number of additional instruments may also be inserted through the system 10. An example of such an instrument includes an elongate tissue engagement tool 74 having an elongate flexible shaft 76 with a removable handle or grip 78 located on its proximal end. The tissue engagement tool 74 may be positioned within an instrument lumen 102 adjacent to instrument lumen 100. The distal end of flexible shaft 76 may include a rotatable helical tissue engager 80 used to temporarily engage and manipulate tissue. The helical tissue engager 80 may further include a number of visual indications or markers near or at the distal end of flexible shaft 76. Examples of tissue engagement tool 74 are described in further detail in U.S. patent application Ser. No. 11/303,521 filed Dec. 16, 2005, which is incorporated herein by reference in its entirety.

In use, tissue manipulation assembly 40 and helical tissue engager 80 may be advanced distally out from elongate body 14 through their respective lumens 100, 102. Tissue engager 80 may be advanced into contact against a tissue surface and then rotated via its proximal handle 78 until the tissue is engaged. The engaged tissue may be pulled proximally relative to elongate body 14 and tissue manipulation assembly 40 may be actuated via its proximally located handle into an open expanded jaw configuration for receiving the engaged tissue.

Additional instruments may also be introduced through elongate body 14, such as conventional endoscopic instruments including graspers, scissors, needle knives, snares, etc., through a corresponding instrument lumen 104. In one example, an endoscopic instrument 82 having a flexible shaft 84 with a manipulatable handle or control 86 at its proximal end and a scissor mechanism 88 at its distal end may be introduced through the elongate body 14 for performing tasks such as cutting of tissue and/or sutures.

To endoscopically visualize procedures and tissue regions of interest, an endoscope or imaging system 90 having a flexible shaft 92 may be introduced into the elongate body 14 via a side port, e.g., Y-Port 96, located along the elongate body 14 and distal to handle assembly 16, as shown in FIG. 1A. Flexible shaft 92 may be advanced through visualization lumen 98 such that its distal end is advanced distally of the elongate body distal end 12 or it may be parked at the terminal opening of the visualization lumen 98 for providing imaging of a procedure. Although shown as an endoscope 90 in this illustration, other variations may include an imaging chip such as a CCD imager integrated into the distal end 12 of elongate body 14. A cable 94 extending from endoscope 90 may be connected to a processor and monitor (not shown) for providing the images.

Endoscope 90 may be introduced directly through handle assembly 16 in other variations; however, positioning the imaging system 90 through a distally located Y-Port 96 relative to handle assembly 16 may allow for a longer length of the shaft 92 to be introduced through visualization lumen 98 into the patient body. As elongate body 14 is advanced into the patient body, e.g., per-orally and into the stomach, the Y-Port 96 remains outside the patient body.

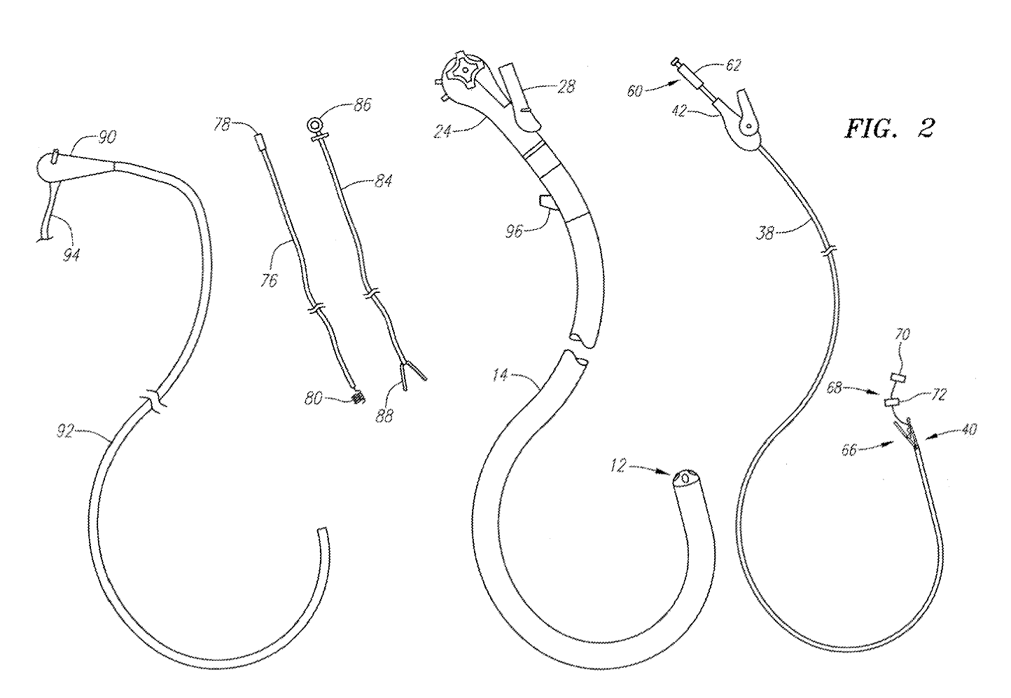

FIG. 2 shows endoscopic manipulation system 10 disassembled into its separate instrument components for illustrative purposes. As seen, the handle 42 of tissue manipulation assembly 40 and its flexible shaft 38 may be removed from elongate body 14. Removable needle deployment assembly 60 with its needle assembly control or housing 62 and its elongate shaft extending through flexible shaft 38 and terminating in needle assembly 66 may also be removed from elongate body 14. Also shown is anchor assembly 68 comprising, e.g., distal tissue anchor 70 and proximal tissue anchor 72, which may be deployed from needle assembly 66 through flexible shaft 38.

Also shown is helical tissue engager 80 disposed upon flexible shaft 76 and endoscopic instrument 88, e.g., endoscopic scissors, disposed upon flexible shaft 84, removed from elongate body 14 and handle assembly 16. Further shown is endoscope 90 with endoscope shaft 92 removed from Y-Port 96.

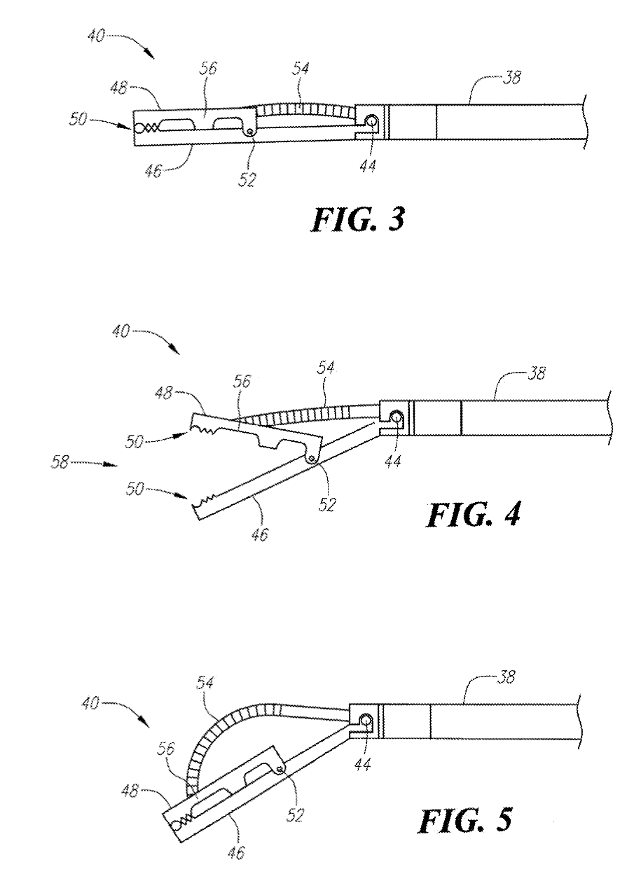

As mentioned above, tissue manipulation assembly 40 is further described in detail in U.S. patent application Ser. No. 11/070,863 filed Mar. 1, 2005 and published as U.S. Pat. Pub. 2005/0251166 A1. An illustrative side view of one example is shown in FIG. 3, which shows assembly 36. The assembly 36 generally comprises a flexible catheter or tubular body 38 which may be configured to be sufficiently flexible for advancement into a body lumen, e.g., transorally, percutaneously, laparoscopically, etc. Tubular body 38 may be configured to be torqueable through various methods, e.g., utilizing a braided tubular construction, such that when handle 42 is manipulated and/or rotated by a practitioner from outside the patient's body, the longitudinal and/or torquing force is transmitted along body 38 such that the distal end of body 38 is advanced, withdrawn, or rotated in a corresponding manner.

Tissue manipulation assembly 40 is located at the distal end of tubular body 38 and is generally used to contact and form tissue folds, as mentioned above. FIG. 4 shows an illustrative detail side view in which tissue manipulation assembly 40 may be seen connected to the distal end of tubular body 38 via a pivotable coupling 44. Lower jaw member 46 extends distally from the pivotable coupling 44 and upper jaw member 48, in this example, may be pivotably coupled to lower jaw member 46 via jaw pivot 52. The location of jaw pivot 52 may be positioned at various locations along lower jaw 46 depending upon a number of factors, e.g., the desired size of the “bite” or opening for accepting tissue between the jaw members, the amount of closing force between the jaw members, etc. One or both jaw members 46, 48 may also have a number of protrusions, projections, grasping teeth, textured surfaces, etc., 50 on the surface or surfaces of the jaw members 46, 48 facing one another to facilitate the adherence of tissue between the jaw members 46, 48.

Launch tube 54 may extend from handle 42, through tubular body 38, and distally from the end of tubular body 38 where a distal end of launch tube 54 is pivotally connected to upper jaw member 48 at launch tube pivot 56. A distal portion of launch tube 54 may be pivoted into position within a channel or groove defined in upper jaw member 48, to facilitate a low-profile configuration of tissue manipulation assembly 40. When articulated, either via launch tube 54 or other mechanism, as described further below, jaw members 46, 48 may be urged into an open configuration to receive tissue in jaw opening 58 between the jaw members 46, 48.

Launch tube 54 may be advanced from its proximal end at handle 42 such that the portion of launch tube 54, which extends distally from body 38, is forced to rotate at hinge or pivot 56 and reconfigure itself such that the exposed portion forms a curved or arcuate shape that positions the launch tube opening perpendicularly relative to upper jaw member 48, as shown in FIG. 5. Launch tube 54, or at least the exposed portion of launch tube 54, may be fabricated from a highly flexible material or it may be fabricated, e.g., from Nitinol tubing material which is adapted to flex, e.g., via circumferential slots, to permit bending.

Once the tissue has been engaged between jaw members 46, 48, a needle deployment assembly 60 may be urged through handle 42 and out through launch tube 54 by introducing needle deployment assembly 60 into the handle 42 and through tubular body 38 such that the needle assembly 66 is advanced from the launch tube and into or through approximated tissue. The needle deployment assembly 60 may pass through lower jaw member 46 via needle assembly opening defined in lower jaw member 46 to pierce through the grasped tissue. Once the needle assembly 66 has been passed through the engaged tissue, a distal and proximal tissue anchor 70, 72 of the anchor assembly 68 may be deployed or ejected on one or opposing sides of a tissue fold for securing the tissue.

Anchor assembly 68 is normally positioned within the distal portion of tubular sheath 64 which extends from needle assembly control or housing 62. Once the anchor assembly 68 has been fully deployed from sheath 64, the spent needle deployment assembly 60 may be removed from assembly 36 and another needle deployment assembly may be introduced without having to remove assembly 36 from the patient. The length of sheath 64 is such that it may be passed entirely through the length of tubular body 38 to enable the deployment of needle assembly 66 into and/or through the tissue.

Because of the number of different instruments and the different types of tools which may be utilized in endoscopic tissue manipulation system 10, tool or instrumentation management is one consideration for the practitioner or practitioners to facilitate efficient surgical and/or endoscopic procedures when performed upon a patient. Additionally, the division of responsibility for instrumentation management between one or more practitioners is highly desirable to ensure patient safety and procedure facilitation. Several device management systems are described in U.S. patent application Ser. No. 12/138,348 (Attorney Docket No. USGINZ05600), filed Jun. 12, 2008, which was previously incorporated by reference herein. The systems described in the foregoing application include trays, stands, tables, clamps, belts, and other supports used to support or hold the endoscopic tissue manipulation system 10 or one or more portions of the system.

Several of the instrument management system embodiments described herein and in the '348 application referenced above facilitate use of the endoscopic access system by the operator in either a “hands on tools” mode with the system retained in the stand or support arm, or a “hand on scope/hand on tool” mode in which the operator holds the handle 24 in one hand and an instrument with the other hand. Those skilled in the art will recognize that the “hands on tools” mode corresponds generally with the manner in which laparoscopic procedures are typically performed, while the “hand on scope/hand on tool” mode corresponds generally with the manner in which endoscopic procedures are performed. Each of these modes of use are facilitated using the instrument management systems described herein. For example, many surgical instrument holders are configured to clamp onto the shaft of a 5 mm or 10 mm instruments. By providing a 5 mm or 10 mm cylindrical post on the handle 24 of an endoscopic access system, the handle 24 may be selectively clamped onto and removed from the instrument holder by the operator. In this way, the operator can simply place the post in the holder and lock it in place to use the system in a “hands on tools” mode, or remove it from the holder and use the system in a “hand on handle/hand on tool” mode.

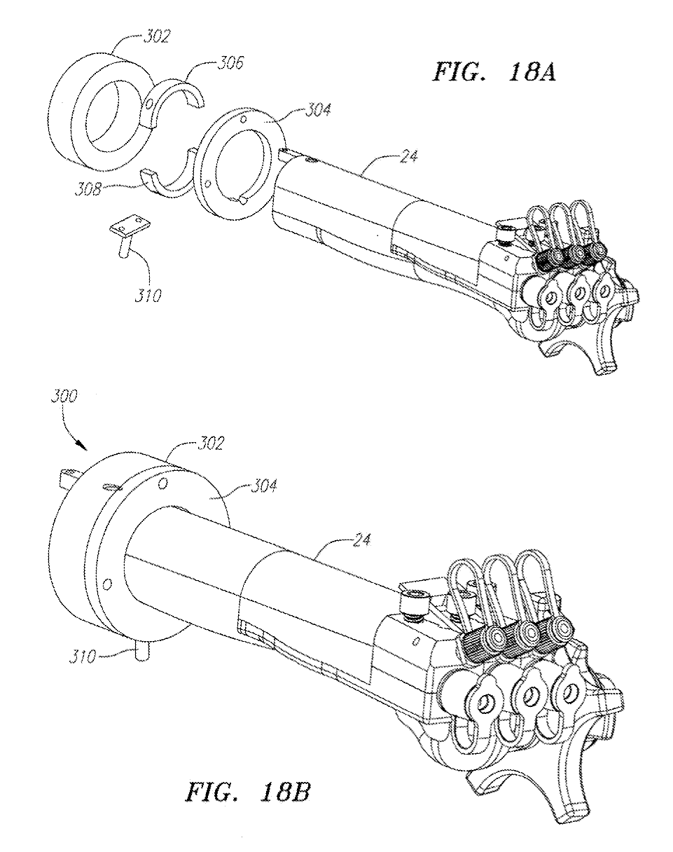

Aside from or in addition to table-mounted or stand-alone instrument supporting members, additional instrument management systems may be employed which a single operator or user may utilize. One example is shown in FIGS. 18A and 18B, which show perspective views of a handle 24 and a rotating clamp mechanism 300 that serves as a functional interface between a support arm (e.g., a stand or other holder) and the endoscopic access system. The clamp 300 includes a generally cylindrical housing 302, a backing plate 304, an upper clamp half 306, and a lower clamp half 308. The housing 302 is generally cylindrical in shape, having a central through hole having a size sufficient to allow the handle 24 to pass therethrough. The housing 302 also includes a channel formed on its inner surface and adapted to receive the upper clamp half 306 and lower clamp half 308, each of which has a generally semi-circular shape to facilitate rotational movement within the housing channel. The backing plate 304 is attached to each of the upper clamp half 306 and lower clamp half 308 and the combined unit is fixed to the outer surface of the handle 24. As a result, the handle 24 is allowed to rotate within the clamp housing 302 while being supported by the clamp mechanism 300. A post 310 is attached to the clamp housing 302. The post 310 has a size and shape that facilitates attachment to a clamp or other mechanism contained on the stand, support arm or other mechanism, thereby providing the ability to mount the endoscopic access system on the stand or support arm while providing free rotation of the handle 24 relative to the stand or support arm.

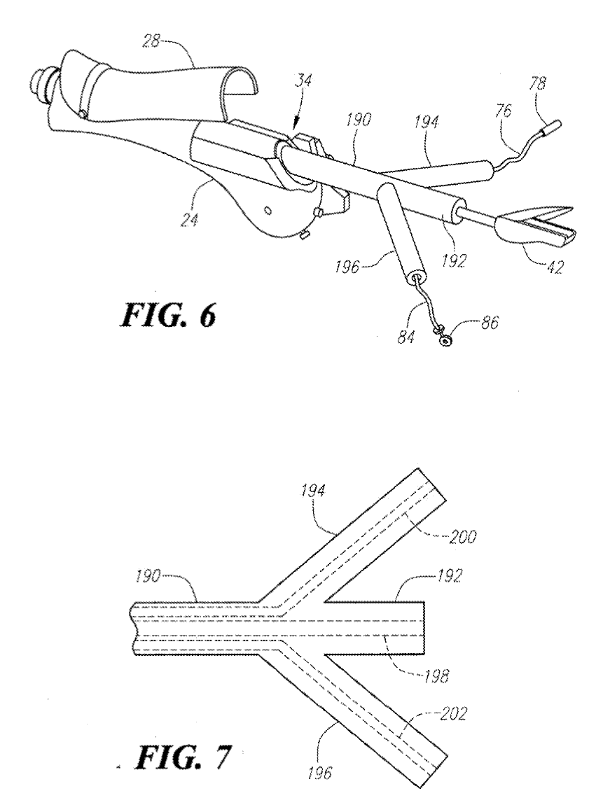

Another instrument management system is shown in FIG. 6, which shows a perspective view of handle 24 having a multi-instrument support arm 190 extending therefrom. Support arm 190 may generally comprise a stiffened multi-lumen channel having a straight support channel 192 extending proximally and one or more angled or curved support channels 194, 196 projecting at an angle from support arm 190. Although two angled support channels are shown in this illustration, additional support arms may be utilized as practicable and as desired depending upon the number of tools advanced through elongate body 14. In the example, handle 42 of tissue manipulation assembly 40 is positioned through the straight support channel 192 while instrument shafts 76, 84 are positioned through their respective support channels 194, 196.

As shown in the partial cross-sectional view of FIG. 7, each support channel may have a corresponding separate lumen defined therethrough. For instance, straight support channel 192 may have instrument lumen 198 defined therethrough, while angled support channels 194, 196 may have respective instrument lumens 200, 202 defined therethrough. Because multi-instrument support arm 190 is relatively stiff, e.g., support arm 190 may be comprised of a metal such as stainless steel or a stiffened polymeric material or plastic, support arm 190 may be engaged to handle 24 and used to support and separate its respective instruments leaving the operator to hold a single handle 24 during a procedure.

In an alternative configuration, portions of or the entire support arm 190 is formed of a relatively flexible material, such as a rubber or polymeric material. The flexibility of the support arm 190 allows instruments having relatively rigid shafts to pass through the instrument lumens 198, 200, 202 despite the presence of any non-linear portions of the lumens. For example, the support arm 190 is sufficiently flexible that the support channels 194, 196 are able to flex in response to the rigid instrument shaft as it passes through any non-linear portions of the lumen.

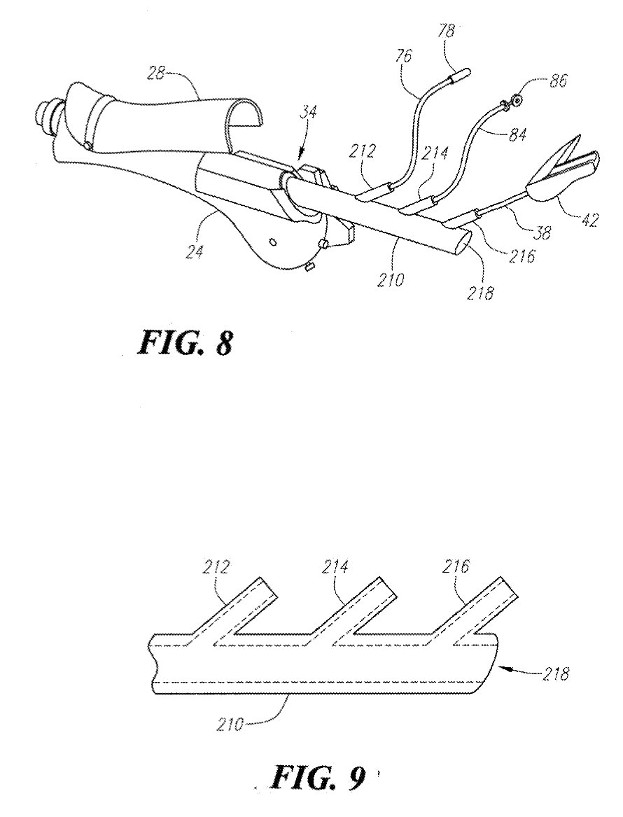

Another example of a multi-instrument support arm 210 is shown in the perspective view of FIG. 8, which illustrates a straight tubular member 210 which defines a lumen therethrough 218 and having one or more angled or curved support channels 212, 214, 216 each defining an instrument lumen therethrough, as shown in the partial cross-sectional view of FIG. 9. In an alternative embodiment, each of the support channels 212, 214, 216 provides access to a separate instrument lumen extending through the support arm 210, the handle 24, and the elongate body 14. In this variation, each of the instruments, positioned through each respective channel, may be supported by the support arm 210 and separated for individual control and manipulation. As above, support arm 210 may be made from a stiff material to enable manipulation of handle 24 while support arm 210 supports the various instruments during a procedure.

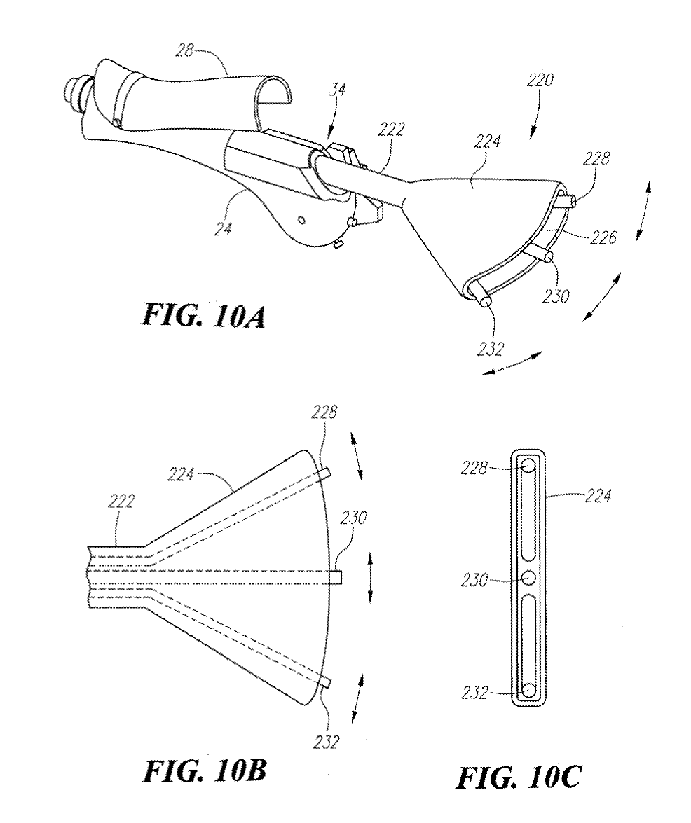

In yet another variation, a pivoting multi-instrument support 220 is illustrated as generally having a support arm 222 with a fanned or angled lumen enclosure 224 extending therefrom, as shown in FIG. 10A. Enclosure 224 may define an open channel 226 within which one or more individual instrument ports 228, 230, 232 may be pivotably positioned, as shown in the top and end views of FIGS. 10B and 10C, respectively. The instruments to be advanced through elongate body 14 may be passed into their respective instrument ports, each of which may be individually pivoted within open channel 226 respect to one another.

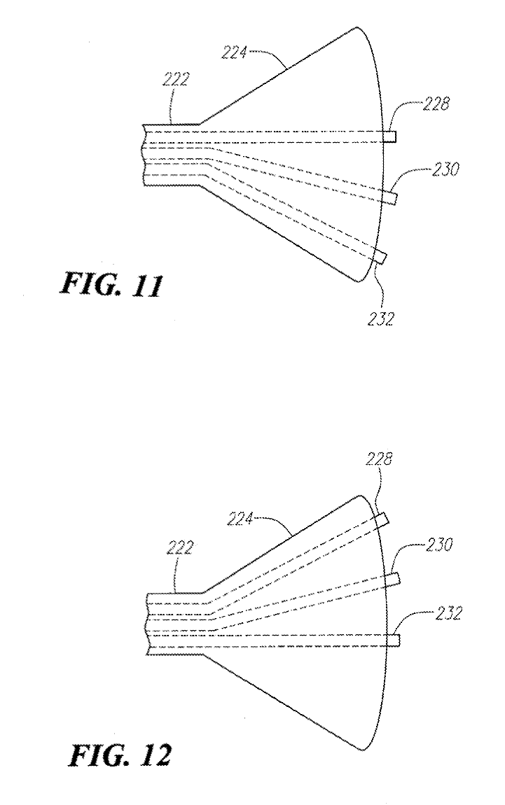

FIGS. 11 and 12 show examples of how each individual instrument port 228, 230, 232 may be pivoted into a straightened lumen to facilitate handling or articulation of an individual instrument positioned within a respective port. For instance, as shown in FIG. 11, instrument port 228 may be pivoted within enclosure 224 to straighten its lumen. If another instrument, which may be positioned within instrument port 232, were to be straightened within enclosure 224, e.g., for withdrawal or advancement, each instrument port may be pivoted within enclosure 224 until the selected port 232 were positioned into its straightened configuration, as shown in FIG. 12.

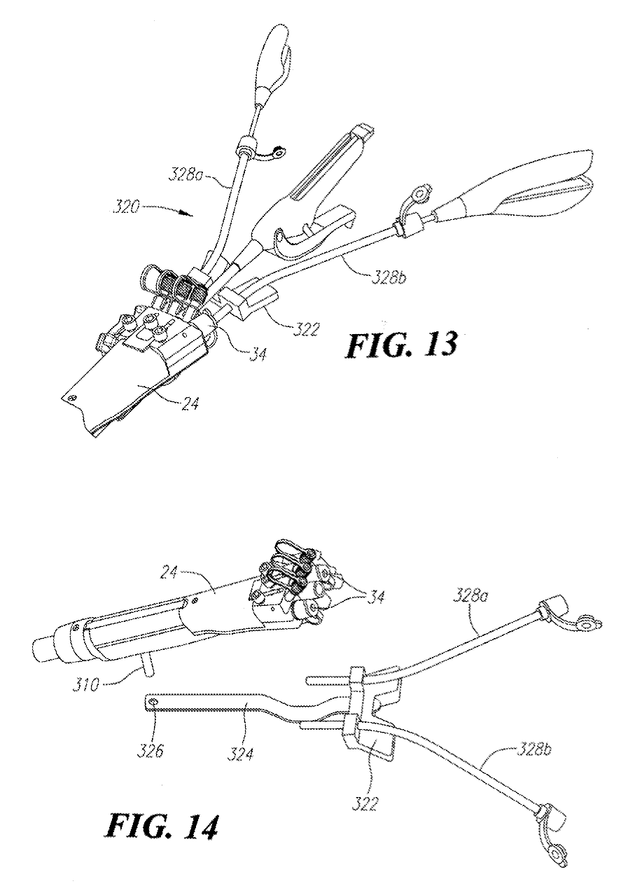

Turning to FIGS. 13 and 14, an alternative multi-instrument support mechanism 320 includes a manifold 322 that is attached to the handle 24 of an endoscopic access system. In the embodiment shown, the manifold 322 includes an elongated tab 324 having a hole 326 that attaches to a post 310 on the handle 24. The manifold 322 supports a plurality of elongated docking sections 328a, 328b, each of which extends from an instrument port 34 of the handle 24. Each docking section 328a, 328b comprises a rigid tube having an elongated straight section adapted to receive a flexible instrument and route the instrument shaft into the respective instrument port 34 and through the handle 24 and elongate body 14 of the endoscopic access system. The docking sections 328a, 328b may optionally include a bend or other feature, such as the bends shown in the embodiment shown in FIGS. 13 and 14. The bends provide a spread alignment of the instruments retained within the docking sections 328a, 328b to thereby reduce or prevent clashing of the instrument handles. The spread alignment may take several optional forms. For example, all of the instruments retained in the docking sections 328 may be extended an equal length beyond the proximal end of the handle 24 and spread in a single plane or in multiple planes. For illustrative purposes, the system shown in FIGS. 13 and 14 illustrates a spread in a single plane but with a central instrument extended a shorter length from the proximal end of the handle 24. In alternative embodiments, the docking sections 328a, 328b are separately positionable so as to provide the user with a desired spread or orientation.

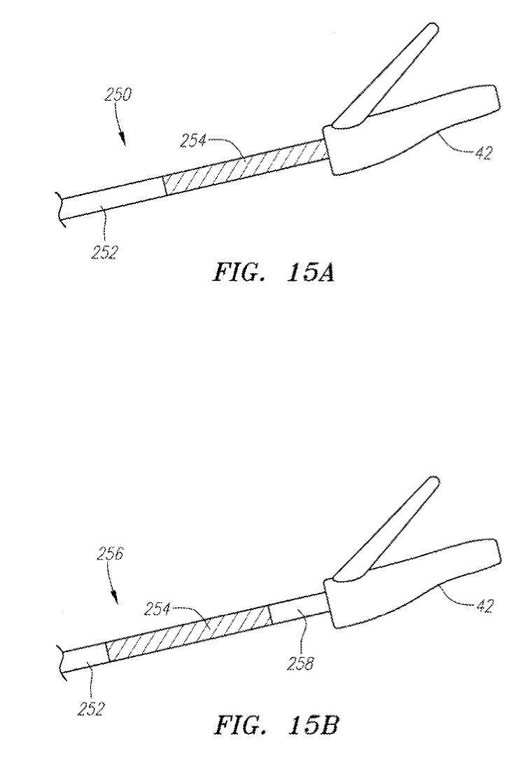

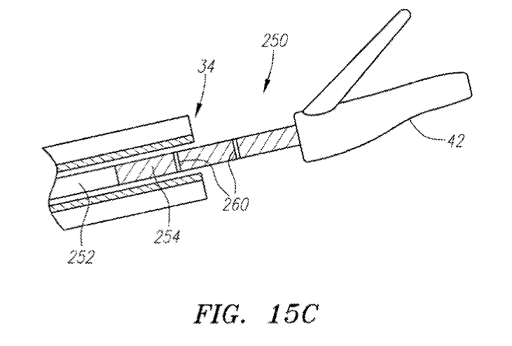

Another method for facilitating instrument management utilizes forming rigid portions of the instrument shafts. An example is shown in the side view of FIG. 15A which illustrates handle 42 and a proximal portion 250 of the elongate shaft of the tissue manipulation assembly 40. The elongate shaft is generally configured as a flexible length 252 so as to traverse through elongate body 14 and within the patient body via endoluminal pathways. A portion of the elongate shaft extending between handle 42 and flexible length 252 may be configured as a rigid section 254. Rigid section 254 may include a rigid sleeve made, e.g., from stainless steel or some other rigid metal or polymer, which is formed over the portion of the shaft extending from handle 42. Alternatively, the rigid portion 254 may be formed integrally with the elongate shaft, e.g., as a section reinforced by woven metallic braids or inserts. Rather than having the rigid section 254 extend directly from handle 42, rigid section 254 may be positioned between two flexible lengths 252, 258, as shown in the rigidized elongate body 256 in FIG. 15B.

In use, the flexible length of elongate shaft 252 may be advanced through a tool port 34 and through handle assembly 16. Rigid section 254 extending from handle 42 may be advanced at least partially into tool port 34, as shown in FIG. 15C, such that handle 42 is supported or held in a linear configuration relative to tool port 34 and handle assembly 16 by the rigid section 254. The absence of rigid section 254 from flexible shaft 252 would allow handle 42 to flex and bend relative to tool port 34 in an uncontrolled manner. In the case where a configuration as shown in FIG. 15B is used, rigid section 254 may be positioned to extend from the entry of tool port 34 to provide some support to handle 42 while the proximal flexible section 258 extending between rigid section 254 and handle 42 may still allow for some limited flexibility in moving or articulating handle 42 in a non-linear manner relative to tool port 34 and handle assembly 16.

Additionally, one or more visual markings or indicators 260 may be provided along the length of rigid section 254, as shown in FIG. 15C. These visual indicators 260 may correspond to the depth which the tissue manipulation assembly 40 has been inserted into the patient body or the length which tissue manipulation assembly 40 has been advanced past the distal end of the rigidizable elongate body 14 within a body lumen of a patient.

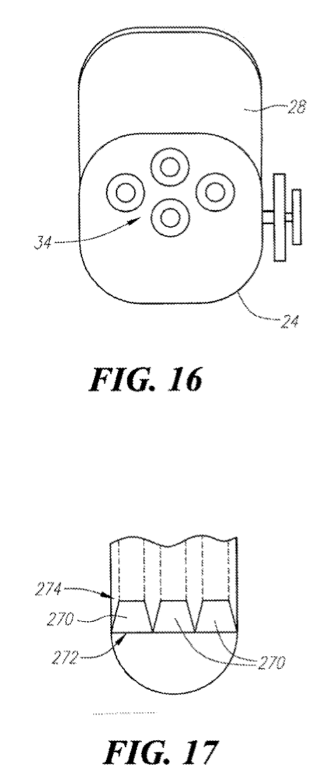

In addition to the various device and instrument management tools and systems described above, tool ports 34 in handle assembly 16 may also be configured to facilitate device management. As shown in the end and top views of handle assembly 16 in FIGS. 16 and 17, respectively, the entry to tool ports 34 may be configured as a tapered instrument port 270. Tools and instruments may be inserted through the enlarged entry 272 and guided into the narrower tool lumen 274 by the narrowing tapered surface of port 270.

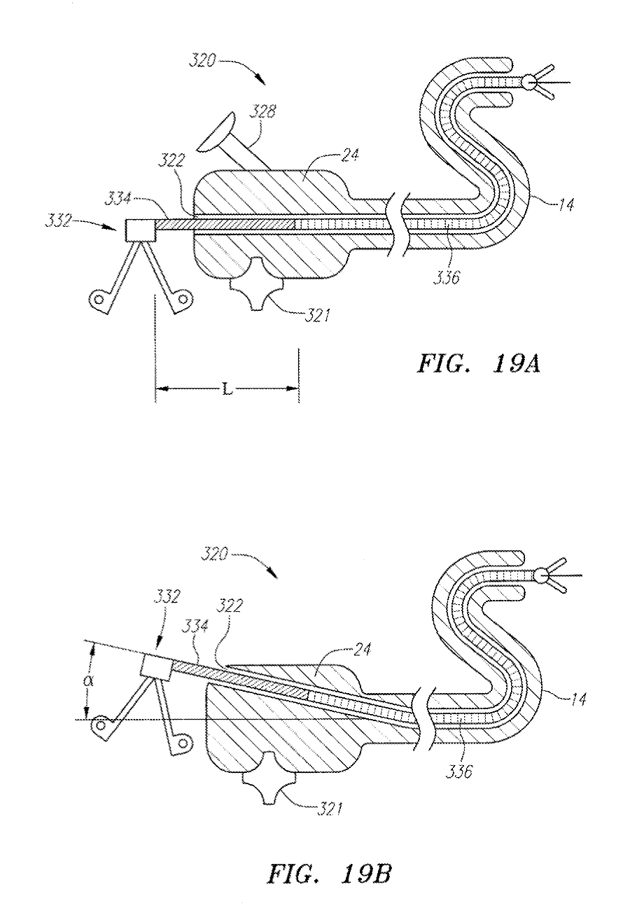

Several of the features of the tools and systems described above in relation to FIGS. 6-17 are further described in relation to FIGS. 19A and 19B, which illustrate the slidable docking feature of an endoscopic access device and a flexible instrument. Referring to FIG. 19A, an endoscopic access device 320 is shown, the device having a handle 24 with an eyepiece 328 and steering controls 321. The device includes an instrument channel 322 extending through the handle 24 that is elongated and substantially straight through at least a proximal section. The instrument 332 includes a shaft having a substantially rigid proximal section 334 and a substantially flexible distal section 336. As described above, the slidable docking interface provided between the instrument channel 322 and the rigid proximal shaft 334 allows the operator to release the instrument, upon which the instrument will remain stably docked within the handle 24 of the access device 320. In several embodiments, the length L of the rigid proximal section 334 of the instrument shaft is no longer than the rigid length of the instrument channel 322 so as not to interfere with the flexibility of the flexible section of the endoscopic access device 320 when the instrument shaft is inserted into the device to its intended extent. The length L of the rigid proximal section 334 should, however, be sufficient to provide additional overlap so that slidable docking occurs (i.e., no backing out to the flexible shaft section 336) during normal operation of the instrument.

As shown in FIG. 19B, the elongated and substantially straight section of the instrument channel 322 extending through the handle 24 need not be in line with longitudinal axis of the flexible section 14 of the endoscopic access device. In the embodiment shown in FIG. 19B, the docking section of the instrument channel 322 is inclined at an angle α relative to the longitudinal axis of the flexible section 14. The flexible portion of the instrument shaft 336 is sufficiently flexible to accommodate the bend created by the differential.

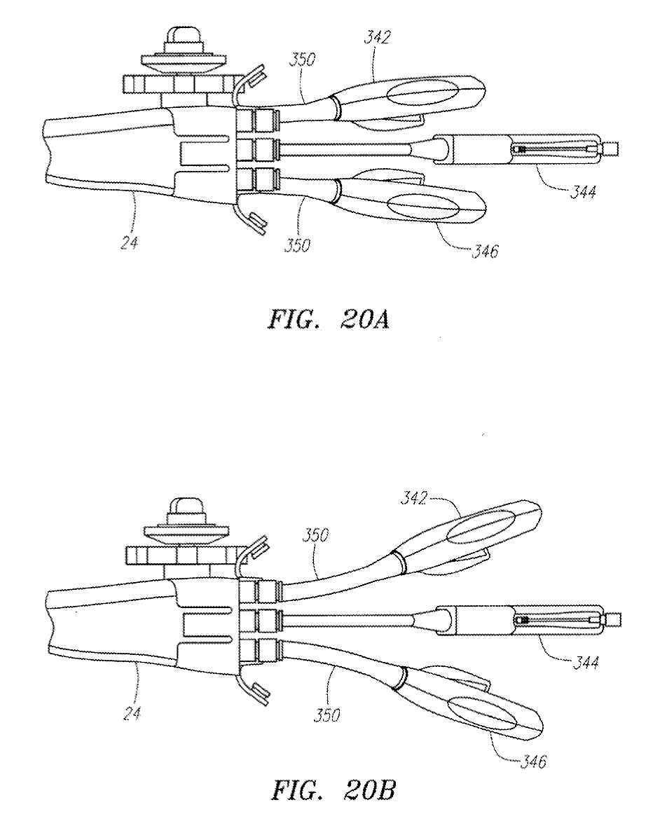

In addition to the other instrument management tools and systems described herein, another mechanism for reducing or eliminating clashing of instrument handles is shown in FIGS. 20A and 20B. An endoscopic device handle 24 includes a plurality of instruments 342, 344, 346 extending from a plurality of instrument ports 34 located on the proximal end of the handle. As shown in FIG. 20B, two of the instruments include a flexible joint 350 located adjacent to the instrument handle between the handle and the rigid portion 334 of the instrument shaft. The flexible joint 350 are sufficiently flexible to allow the handle to be bent away from handles of other instruments received in the device while retaining sufficient rigidity to prevent drooping. In this manner, the handles of adjacent instruments may be flexed apart rather than clashing.

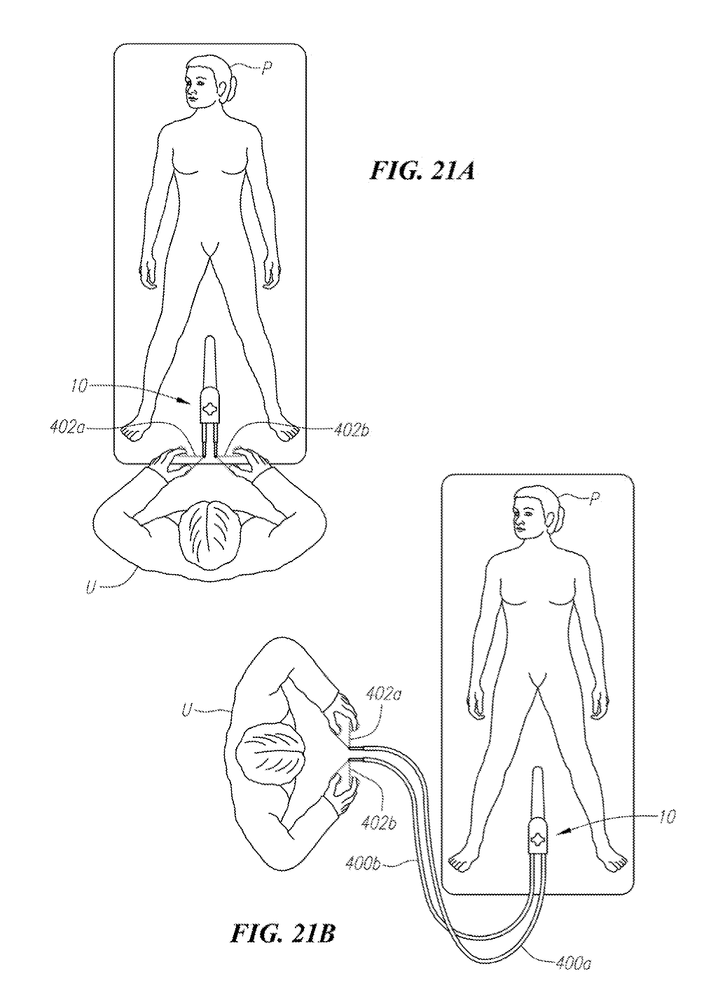

Turning next to FIGS. 21A-B, another embodiment of an endoscopic instrument management system is shown. In the embodiment, one or more selectively attachable and detachable instrument management manifolds 400 allow the user to use the endoscopic manipulation system 10 either with a compact handle (such as when not using multiple instruments), but then to attach the manifold to obtain a spread lumen configuration (such as when using multiple tools). Preferably, the endoscopic manipulation system 10 is held in a fixed or positionable stand when the tool spreading manifold is in use and multiple tools are in use so as not needing a hand to hold the system.

The instrument management manifolds 400 shown in FIG. 21B are capable of being configured to take on a desired shape or orientation. For example, as shown in FIGS. 21A-B, a female patient P is in the lithotomy position and the endoscopic manipulation system 10 is placed in a location relative to the female patient P to have the distal section of the system be introduced through the vagina in order to perform a gynecological examination or therapeutic procedure. In the embodiment shown in FIG. 21A, the instruments 402a, 402b are introduced into the system 10 by insertion directly into tool ports 34 in the handle 24. This orientation requires that the physician or other user be located in the position shown in FIG. 21A, between the legs and toward the feet of the patient P. In the embodiment shown in FIG. 21B, the instruments 402a, 402b are introduced into the system 10 by way of a pair of instrument management manifolds 400a, 400b that are attached to the proximal side of the handle 24. Each manifold 400a, 400b is attached at a first end to a port 34 of the handle 24. The manifold has a generally “U”-shape or other curved shape that extends proximally from the handle 24 and then generally up and laterally to the side of the patient P. This orientation allows the physician or other user U to be located at the flank of the patient P in a conventional laparoscopic stance with the monitor at the feet of the patient P and the physician facing the monitor. In some circumstances, this positioning may be preferred to provide advantageous instrument access, comfort and/or freedom of movement of the physician, or improved monitor and/or patient visualization and control of the instruments for the physician.

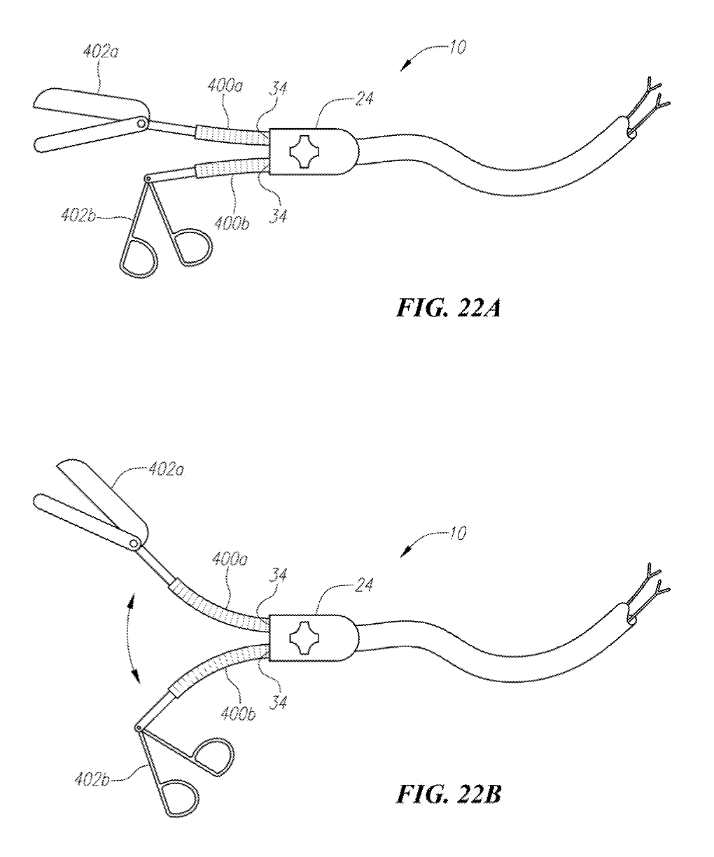

Turning to FIGS. 22A-B, the manifolds 400a, 400b are generally tubular structures defining a lumen that extends through the length of the manifold. In some embodiments, the manifold is formed of a substantially rigid material that is resistant to bending or other deformation, such as stainless steel. In other embodiments, such as shown in FIGS. 22A-B, the manifolds 400a, 400b are formed of a material that is capable of bending or other deformation under manipulation by the physician or other user, such as a semi-rigid metallic or polymeric material. Examples of materials suitable for the malleable manifold structures include interlocking rolled metal structures used in conventional microphone stand gooseneck devices, or Loc-Line® modular hose system materials available from Lockwood Products, Inc. of Lake Oswego, Oreg. The Loc-Line® system products include a plurality of press-fit jointed structures defining a central lumen therethrough.

In some embodiments, the instrument management manifold 400 is transformable so that the relative positions of the lumens may be altered. For example, a manifold 400a, 400b having the jointed or other malleable structure is able to be bent or otherwise deformed to meet specific procedural needs. For example, the lumens defined by the manifolds 400a, 400b may be positioned straight and relatively close together during insertion of tools through the manifolds and/or the endoscopic manipulation system 10 (see, e.g., FIG. 22A), and then bent or deformed to a working position in which the manifolds 400a, 400b are no longer straight (see, e.g., FIG. 22B).

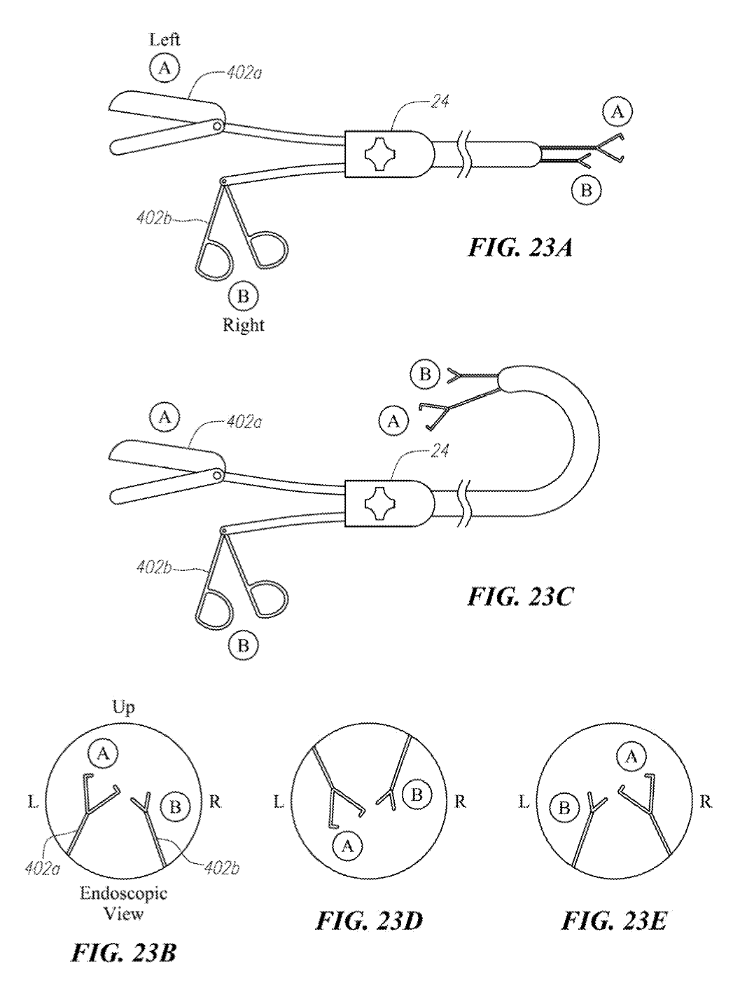

Positionable or bent manifold lumens also are advantageous with regard to the relative hand positions of the clinician manipulating the tools or instruments 402 and what is displayed on a visualization monitor. A phenomenon of “switching” occurs when an endoscopic delivery device is steered into a retroflexed position. The retroflexed position of the device causes the image to turn upside down and reversed. For example, compare FIGS. 23A-B in which the delivery device has a generally straight orientation, with FIGS. 23C-E in which the delivery device is steered into a retroflex position. The endoscopic view shown in FIG. 23D is reversed and upside down relative to the endoscopic view shown in FIG. 23B. To correct the image, the endoscope (located in one of the working lumens of the delivery device) is first rotated 180 degrees in order to make the image no longer appear upside down. Next, after the upside down correction is made, the instruments 402a, 402b extending through the access device will appear on the opposite side of the screen relative to the hands being used to manipulate the instruments 402a, 402b, as shown in FIG. 23E. This “switching” effect will frequently cause disorientation to the physician or other user of the device.

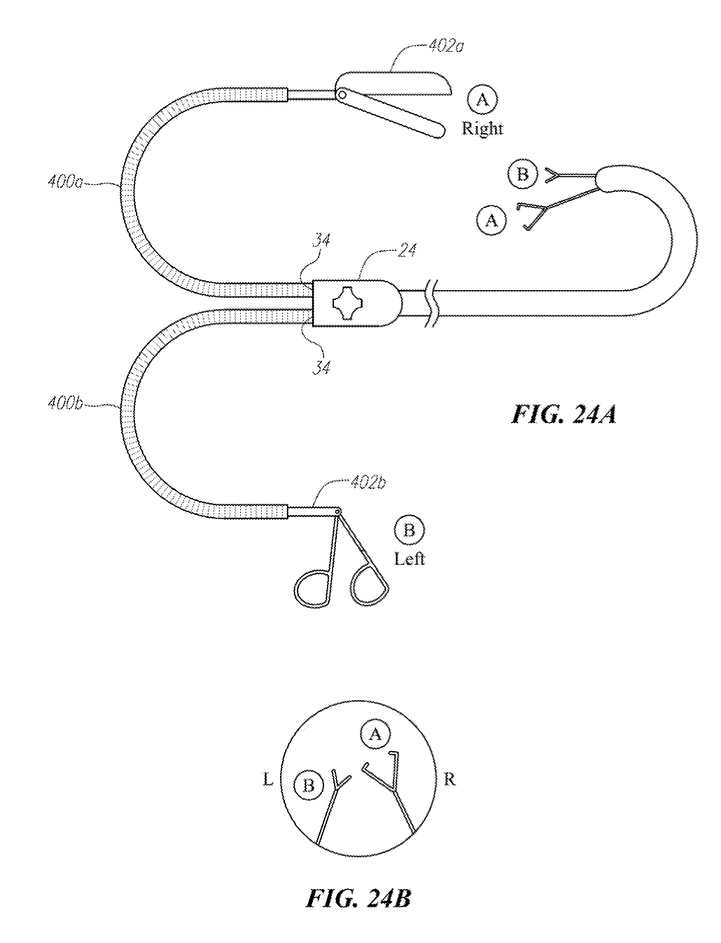

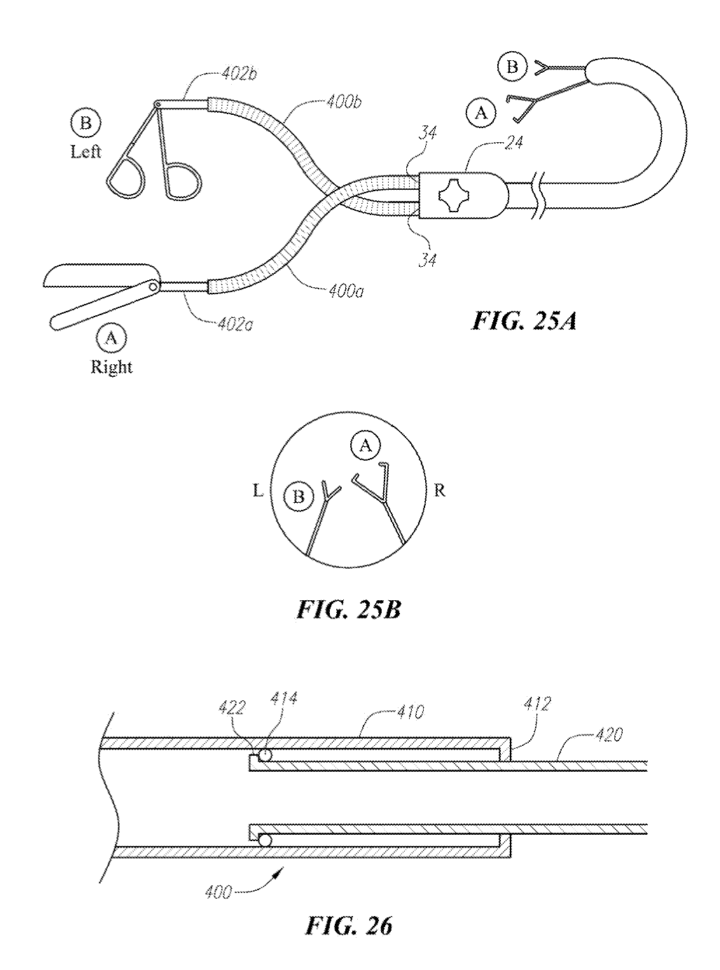

The “switching” phenomenon is corrected using the instrument management manifolds 400 in the following ways, as shown in FIGS. 24A-B and 25A-B. The instrument management manifolds 400a, 400b are curved or deformed into a retroflexed orientation relative to the exit ports 34 of the handle 24 of the delivery device 10. This puts the instruments 402a, 402b back into the correct visual and spatial configuration. See FIGS. 24A-B. Alternatively, the instrument management manifolds 400a, 400b are crossed or crossable with a similar result. See FIG. 25.

In some embodiments, the endoscopic instrument manifold 400 has a construction that allows it to telescope. See FIG. 26. For example, instead of having a rigid telescoping interface, the manifold 400 has a construction of nesting or accordionizing tubing, including a first tube 410 that is attached (directly or indirectly) to the handle 24 of the access system 10, and a second tube 420 that is slidable within and extends proximally of the proximal opening of the first tube 410. The first tube 410 includes an inwardly directed flange 412 formed on or attached to its proximal end, and the second tube 420 includes an outwardly directed flange 422 formed on its proximal end. The flanges 412, 422 prevent the first tube 410 and second tube 420 from becoming separated from one another. An o-ring 414 seals the tubes relative to one another. In other embodiments, more tube sections are used and/or the relative diameters of the tubes are reversed to allow the first tube 410 to telescope within the second tube 420. Other variations are also possible.

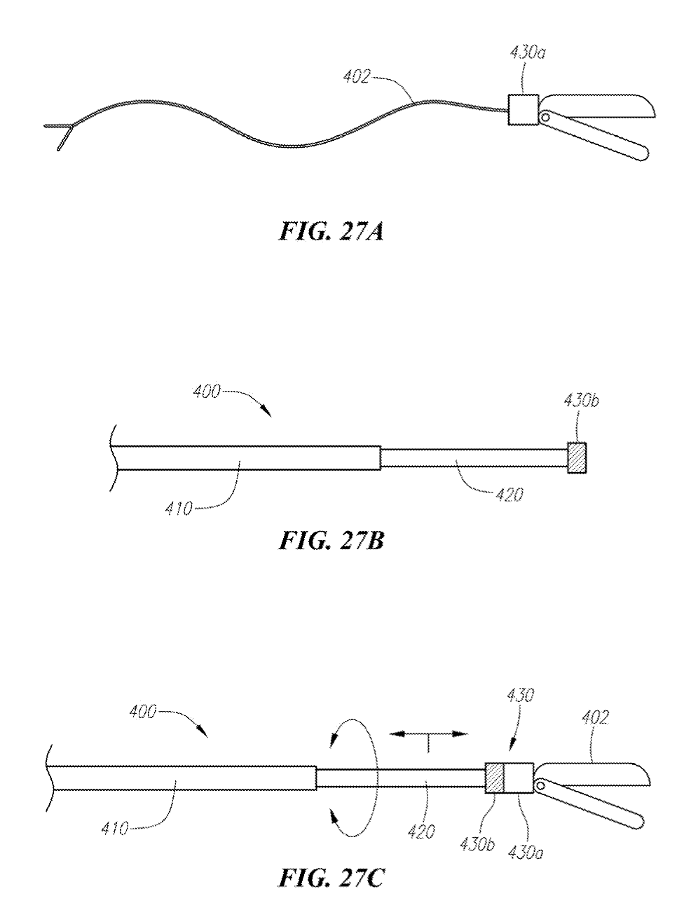

In still other embodiments, the instrument 402 inserted into a manifold 400 is adapted to interlock with the manifold 400 entrance, preferably at the instrument handle. See FIGS. 27A-C. For example, a handle interlocking portion 430a is provided on the shaft or handle of the instrument 402, and a mating manifold interlocking portion 430b is provided on the proximal opening of the manifold 400. The interlocking portions 430a, 430b are configured to mate and/or interlock to form an interlocking member 430 in which the instrument 402 is selectively attached to the proximal tube 420 of the manifold 400. Advancement, retraction, and rotation would then be allowed and supported by the telescoping manifold 400.

The foregoing descriptions of instrument management tools and systems includes descriptions of several components (and embodiments of components) that may be used in a standalone manner or in combination with other components. For example, a preferred embodiment of an instrument management system suitable for use with the endoscopic tissue manipulation system 10 shown in FIG. 1A includes a support stand having a base that is attachable to a bed rail or other fixed location, a first support arm having a clamp or other fixture attachable to the handle 24 of the endoscopic access device, and a second support arm that is attachable to a handle of the endoscope 90. The first support arm and second support arm of the support stand are configured to be selectively fixed in place or to have effectively free range of motion, such as may be provided by having one or more ball joints or other pivotable connections that allow the user to selectively fix or release the system. Alternatively, the second support arm comprises a boom that is held in a fixed relationship to the first support arm, thereby allowing movement of the endoscopic access device and the endoscope 90 as a single unit. In the embodiment, a holder interface, such as a rotating clamp 300 is used to detachably attach the handle 24 to the first support arm via a post 310, thereby providing a rotational movement capability between the handle 24 and the support stand. Another holder interface, such as a C-clamp that is detachable from the second support arm, may be used to attach the endoscope 90 to the second support arm. Further, the endoscopic access device includes a plurality of instrument lumens that support slidable docking of instruments in the handle 24, with one or more of the instruments living a rigid proximal shaft section 254.

Although a number of illustrative variations are described above, it will be apparent to those skilled in the art that various changes and modifications may be made thereto without departing from the scope of the invention. Moreover, although specific configurations and applications may be shown, it is intended that the various features may be utilized in various combinations and in various types of procedures as practicable. It is intended in the appended claims to cover all such changes and modifications that fall within the true spirit and scope of the invention.

What is claimed is:

1. An endoscopic instrument management system, comprising:
an endoscopic access device having a proximal handle and an elongated flexible member extending from the handle and defining a first lumen extending through at least a portion thereof, the handle defining an entry port in communication with the first lumen of the elongated flexible member, the elongated flexible member having a length sufficient such that a distal end of the elongated member may be inserted into the body of a patient and advanced to a target location within the body of the patient while the handle remains outside of the body of the patient;
an instrument manifold removably coupled to the handle of the endoscopic access device, the instrument manifold defining an instrument lumen in communication with the entry port of the handle, the instrument manifold having a generally “U” shape; and
a flexible instrument extending through the instrument lumen of the instrument manifold, the entry port of the handle, and the first lumen of the elongated flexible member.

2. The endoscopic instrument management system of claim 1, wherein said flexible instrument comprises an instrument handle and an instrument shaft, with the instrument shaft including a rigid proximal section and a flexible distal section.

3. The endoscopic instrument management system of claim 2, wherein said instrument manifold includes a first tube and a second tube, with the second tube telescoping within the first tube.

4. The endoscopic instrument management system of claim 3, further comprising a first interlock member on the instrument manifold and a second interlock member on the instrument shaft, with the first and second interlock members being adapted to selectively interlock to thereby attach the instrument shaft to the instrument manifold.

5. The endoscopic instrument management system of claim 1, further comprising:
a second instrument manifold removably coupled to the handle of the endoscopic access device, the second instrument manifold defining a second instrument lumen in communication with a second entry port of the handle, the second instrument manifold having a generally “U” shape; and
a second flexible instrument extending through the second instrument lumen of the second instrument manifold, the second entry port of the handle, and a second lumen of the elongated flexible member.
